(19) United States
(12) Patent Application Publication
(10) Pub. No.: US 2019/0350909 A1
(43) Pub. Date: Nov. 21, 2019

(54) COMPOSITIONS AND METHODS FOR MODULATING HAIR GROWTH

(71) Applicants: William E. Lowry, Los Angeles, CA (US); Matilde Miranda, Los Angeles, CA (US)

(72) Inventors: William E. Lowry, Los Angeles, CA (US); Matilde Miranda, Los Angeles, CA (US)

(21) Appl. No.: 16/415,822

(22) Filed: May 17, 2019

Related U.S. Application Data
(60) Provisional application No. 62/672,935, filed on May 17, 2018.

(51) Int. Cl. A61K 31/436; A61K 8/49; A61K 9/00; A61P 17/14; A61Q 7/00

(52) U.S. Cl. CPC A61K 31/436; A61K 8/4933; A61K 9/0019; A61P 17/14; A61Q 7/00; A61K 9/0014

(57) ABSTRACT

The present disclosure relates to pharmaceutical compositions and methods for inhibiting Pde, promoting hair growth, and treating hair growth disorders, such as baldness or alopecia.

### RELATED APPLICATIONS

This application claims the benefit of U.S. Provisional Application No. 62/672,935, filed May 17, 2018, the contents of which are fully incorporated by reference herein.

### STATEMENT REGARDING FEDERALLY SPONSORED RESEARCH

This invention was made with government support under Grant Number AR070245, awarded by the National Institutes of Health. The government has certain rights in the invention.

### BACKGROUND

Hair follicle stem cells (HFSCs) undergo successive rounds of quiescence (telogen) punctuated by brief periods of proliferation correlating with the start of the hair cycle (telogen-anagen transition). Proliferation or activation of HFSCs is well known to be a prerequisite for advancement of the hair cycle. Despite advances in treatment options, baldness and alopecia continue to be conditions that cannot be successfully treated in all individuals. Some of the existing treatments are inconvenient for users, others require surgical intervention or other invasive procedures. Additional therapies are needed.

### SUMMARY OF THE INVENTION

In certain aspects, the present disclosure provides methods of promoting hair growth or treating hair growth conditions or disorders such as baldness or alopecia, comprising administering to a patient a compound or composition as disclosed herein.

### BRIEF DESCRIPTION OF THE DRAWINGS

FIG. 1A depicts a pathological examination of hair cycle stage at various time points, where D50 indicates 50 days after birth of the animal. The first morphological signs of a telogen-anagen transition occurs at D63. Immunohistochemistry with the indicated stains shows the timing of activation of Creb (pCreb), the location of HFSCs (Sox9), and proliferation (Ki67). This analysis showed that pCreb expression was highest during the telogen-anagen period (D63-D70), coinciding with stimulation of proliferation, assessed by staining for Ki67.

FIG. 1B depicts the gene expression profiling from HFSCs at telogen or telogen-anagen transition and shows a distinct pattern for Pde4b. This indicates that while most Pde levels do not change during this transition, Pde4 was markedly decreased during the telogen-anagen transition.

FIG. 2A shows that stimulation of Creb signaling can accelerate the hair cycle. Mice were shaved at D45 and treated every 48 hours with vehicle, or vehicle+the indicated treatment to stimulate Creb activity. In vehicle-treated animals, pigmentation and hair growth did not start until at least D76, whereas Pde inhibitors (#44 and/or #61) or cAMP-stimulation accelerated this process by up to 14 days. Conversely, treatment with a PKA inhibitor (H89) slowed hair cycle progression.

FIG. 2B shows representative mouse photos of control and treated conditions. Respective skin section and hair follicle images from control and treated mice—as detected by hemotoxylin and eosin staining—are also shown. The topical treatment of mice with Pde inhibitors (#44, #61) accelerates reentry into anagen; topical treatment with PKA inhibitor (H89) delays reentry into anagen as assessed by morphological changes of the hair follicle.

FIG. 2C depicts a cross section of hair follicles after long term treatment with certain compounds. Immunohistochemistry for HFSC/general stem cell markers and associated behavior: Sox9 is a stem cell marker; PCNA is a proliferation marker; pCreb is a marker for active Creb signaling (active HFSCs). Scale bar=25 uM. Topical treatment with Pde inhibitors (#44, #61) accelerated reentry into anagen; and topical treatment with a PKA inhibitor (H-89) delayed reentry into anagen.

FIG. 3A shows pathological examination of hair follicles after 48 hours of the treatment as described in Example 1, prior to macroscopic observation of hair growth.

FIG. 3B shows the effect of acute stimulation of Creb signaling on HFSCs. Immunohistochemistry for the indicated antigens showed activation of Creb (pCreb) in HFSCs (and IFE) in response to topical administration of #44, cAMP, and forskolin—but not with H89, a Pka inhibitor. Immunohistochemistry for HFSC/general stem cell markers and associated behavior: Sox9 is a stem cell marker; PCNA is a proliferation marker. Creb activation was assessed by pCreb and cFos (target get) expression. Scale bar=25 uM.

FIG. 4A shows that Creb activation drives stimulation of Ldh activity. This assay showed that Pde inhibition has a robust effect on Ldh activity in treated skin, while Pka inhibition did not. Epidermis was isolated from animals treated with the indicated compounds, lysed, and characterized for relative Ldh activity. Samples from animals treated with compound #44 and cAMP displayed high Ldh activity, regardless of treatment in male or female animals, while samples treated with H89 did not demonstrate altered Ldh activity. Example 1 further describes the methods by which these samples were analyzed.

FIG. 4B depicts metabolomics analyses of epidermis isolated from animals treated with the indicated compounds. Example 1 further describes the methods by which these samples were analyzed. This analysis demonstrated that most glycolytic metabolites were induced by Pde inhibition, as would be expected if Creb is able to stimulate the production of glycolytic enzymes.

FIG. 4C shows western blots of samples from animals treated as indicated for Creb activation (pCreb) and Ldha protein, which confirm the effect of Pde inhibition, forskolin treatment, cAMP treatment, and inhibition of Creb by H89. Example 1 further describes the methods by which these samples were analyzed.

FIG. 4D shows RT-PCR results for Ldha transcript in epidermis treated for 1 week; forskolin and cAMP treatment caused high levels of expression. Example 1 further describes the methods by which these samples were analyzed.

FIG. 4E shows the effects of cAMP or forskolin on a mouse follicle. Mice were treated topically with Creb-related compounds for 1 week. Fresh skin tissue was harvested, sectioned, then subjected to an LDH assay—used to assess lactate dehydrogenase activity in situ or via plate reader subjected to 475 nm absorbance measurements. Acute stimulation of Creb-dependent signaling stimulated glycolytic activity in HFSCs and in the IFE. Example 1 further describes the methods by which these samples were analyzed.

FIG. 4F shows LDH plate reader assay analysis of protein lysate from fresh skin tissue that was treated with forskolin or cAMP for 1 week. Activity levels of Ldh were higher in the groups treated with forskolin and cAMP at 1 week as compared to the control group. Example 1 further describes the methods by which these samples were analyzed.

### DETAILED DESCRIPTION OF THE INVENTION

The skin and hair protect from harmful environments but require constant maintenance throughout a coated animal's lifetime. Hair follicle stem cells (HFSCs) cycle through periods of growth (anagen), regression (catagen), and rest (telogen). HFSCs located in the stem cell-containing compartment called the bulge have been shown to be the cells responsible for growing hair shafts de novo, contributing to wound healing, and are the cells of origin for squamous cell carcinoma (SCC). Previously, it has been demonstrated that HFSC quiescence is a tumor suppressor mechanism; the initiation of SCC by HFSCs requires active hair cycling.

Growth factor signaling pathways are known to act either negatively or positively on HFSCs to regulate their activation or quiescence. Several lines of evidence point to a role for G-protein-coupled receptor (GPCR) signaling in regulation of the hair cycle. GPCR signaling typically leads to activation of adenyl cyclase which regulates levels of cAMP, and ultimately leading to activation of the Creb transcription factor.

Although GPCR/cAMP/Creb signaling is key in many developmental processes and the target of many pharmaceuticals in clinical practice, there has been no evidence that this pathway acts directly on HFSCs or could be used to modulate their growth cycle. HFSCs are known to specifically express Lgr5, a seven-pass transmembrane receptor, yet there is no evidence that signaling through this receptor acts through a canonical pathway whereby heterotrimeric g-proteins activate adenylyl cyclase to produce cAMP, which then activates PKA to phosphorylate Creb.

It has been proposed that Lgr5 signals through the Wnt pathway to activate HFSCs and drive the hair cycle. Herein is evidence that canonical cAMP/Creb signaling can regulate HFSC activation and the hair cycle. Furthermore, Creb activation is coordinated through the hair cycle and coincident with HFSC activation. Stimulation of cAMP signaling through inhibition of Phospodiesterase (Pde) was sufficient to stimulate HFSCs and accelerate initiation of the hair cycle. Conversely, inhibition of PKA, which abrogates Creb activation, dramatically slowed the hair cycle. Also disclosed herein is evidence that Creb activation stimulates transcription and translation of Ldha enzyme, which is known to stimulate HFSC activation. These data show that cAMP/Creb signaling regulates the hair cycle through regulation of lactate/pyruvate metabolism.

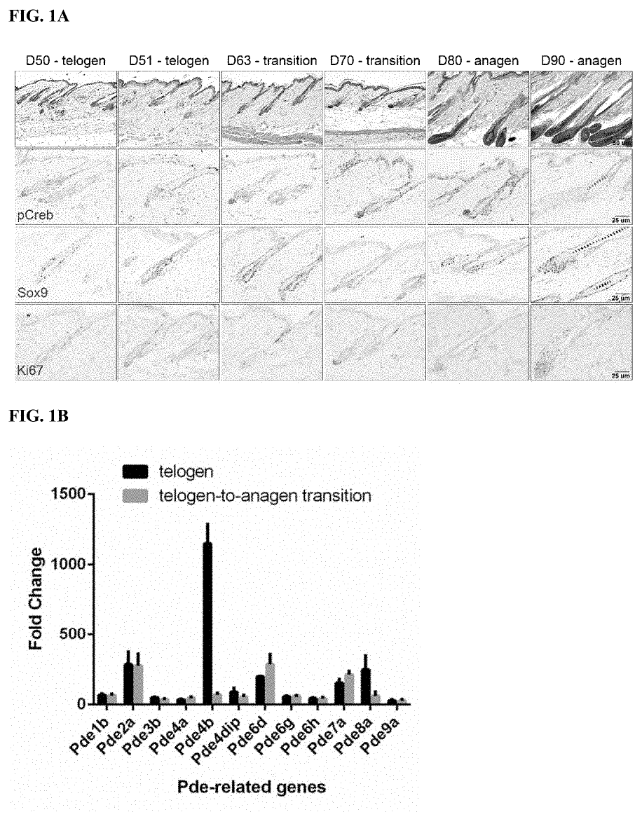

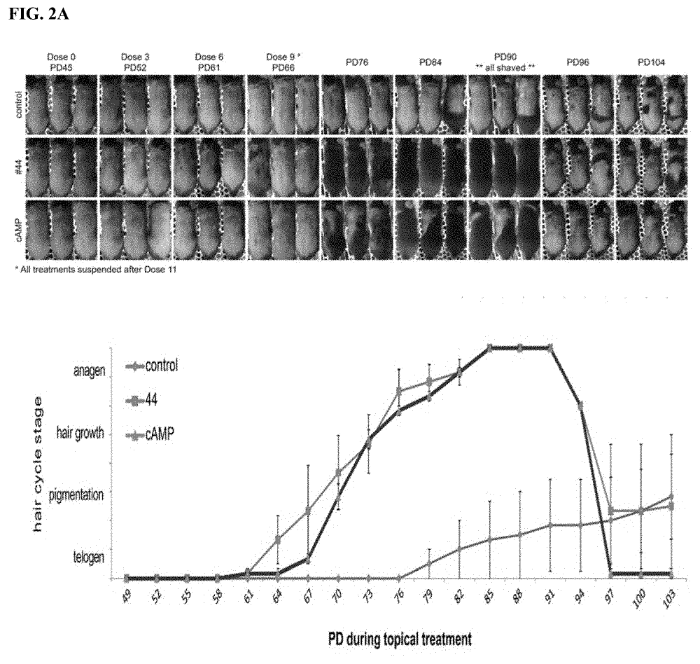

cAMP/Creb signaling has previously been implicated in murine models of Squamous Cell Carcinoma, and tumorigenesis and is thought to depend on activation of HFSCs in this model. It was first investigated whether cAMP/Creb signaling could play a role in HFSC activation or in the hair cycle. The activation of cAMP/Creb signaling across the hair cycle was measured in HFSCs. Immunostaining with an antibody that recognizes the phosphorylated form of Creb was used to identify activation of Creb. This analysis showed that pCreb expression was highest during the telogen-anagen period, coinciding with stimulation of proliferation and assessed by staining for Ki67 (FIG. 1A).

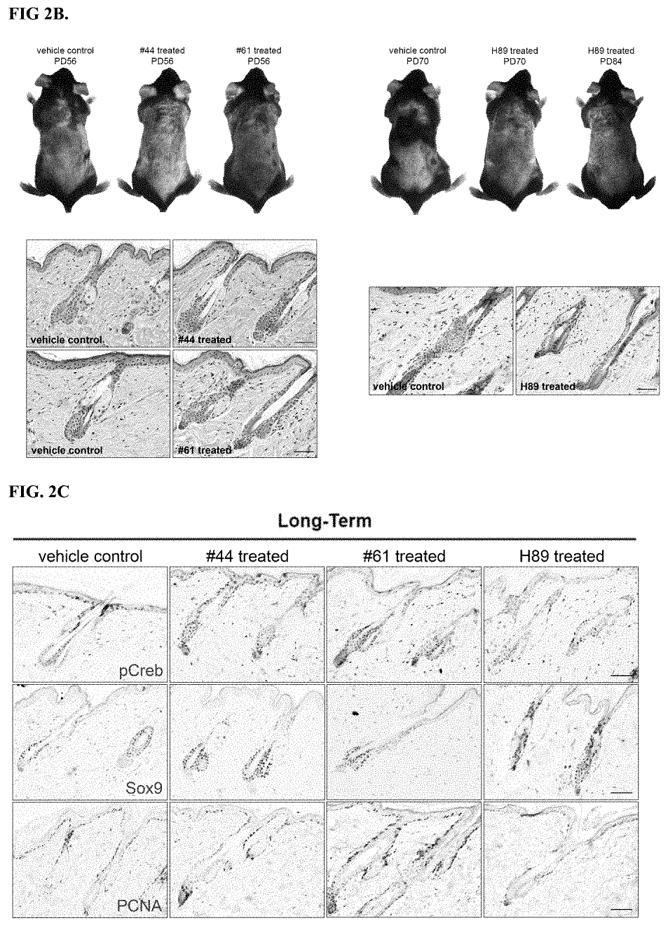

The phosphorylation of Creb occurs through activation of Protein Kinase A (Pka) which is regulated by the concentration of intracellular cAMP. cAMP can be degraded through the action of Phosphodiesterase enzymes (Pde). A previous gene expression study of HFSCs during telogen or telogen-anagen transition was reevaluated to focus on the expression of Pde proteins. This analysis suggested that while most Pde levels do not change during this transition, Pde4 was markedly decreased during the transition phase of the hair cycle (FIG. 1B). This could suggest that induction of pCreb could be the result of loss of Pde activity and therefore stabilization of cAMP levels to activate Pka. To determine whether Pde inhibition is sufficient to activate pCreb and potentially affect the hair cycle, we topically treated mice during telogen with several Pde inhibitors (#44, #61, #62, structures shown in Table 1). Topical Pde inhibition during telogen appeared to strongly accelerate the hair cycle (FIG. 2A, FIG. 2B). Conversely, topical application of H89, an inhibitor of Pka—which should therefore block pCreb accumulation—slowed the hair cycle dramatically (FIG. 2A, upper histogram; FIG. 2B). Because Pde inhibition is known to increase concentrations of cAMP, we also investigated whether directly increasing cAMP concentration through topical application could also affect the hair cycle. cAMP administration stimulated the hair cycle with a similar time course as Pde inhibition (FIG. 2A, lower histogram). Hair follicles treated with phosphodiesterase inhibitors #44 and #61 showed accelerated hair growth as detected by macroscopic darkening of the treated dorsal area, emerging hair shafts, and by elongated hair follicle size as shown with hemotoxylin and eosin staining (FIG. 2B). Long-term phosphodiesterase inhibitor treatment yielded more pCreb positive cells in the bulge and epidermis, and coincided with an upregulation of proliferation indicated by PCNA in many parts of the epidermis and in the HFSC bulge niche (FIG. 2C), while total stem cell amounts (indicated by Sox9 staining) remained the same across all conditions.

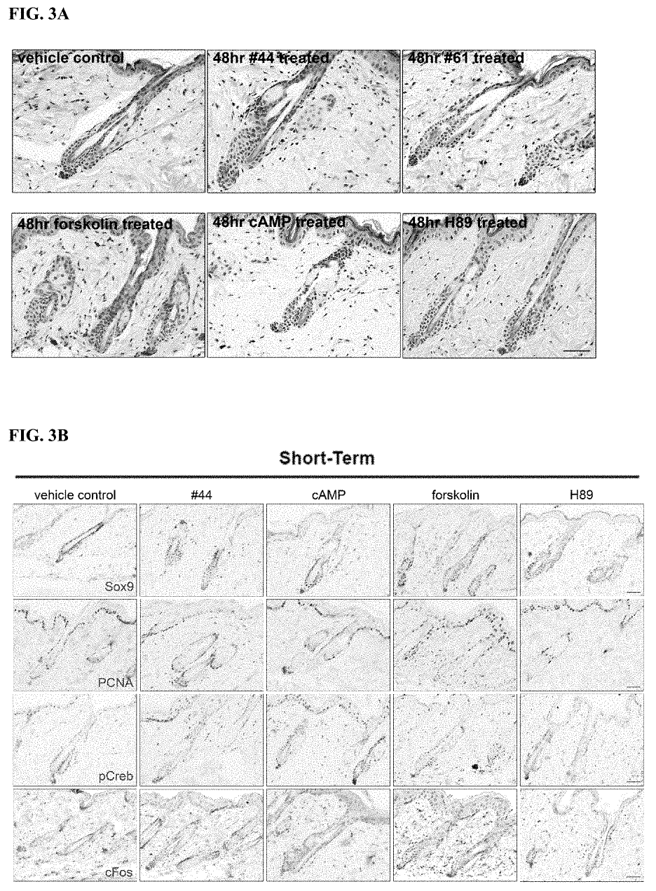

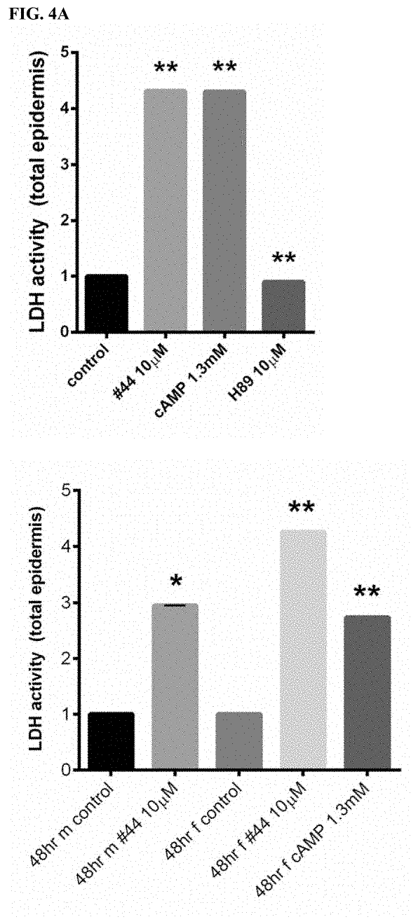

To determine the acute effect of elevated cAMP levels in the skin, immunohistochemistry was performed for markers of cAMP/Creb signaling, HFSCs, and proliferation. Changes in overall morphology was not obvious with 48 hr treatment (FIG. 3A), as expected due to a short treatment window. However, with 48 hours topical treatment, it was clear that elevation of cAMP through direct application of the molecule; by inhibition of Pde activity; or stimulating intracellular cAMP levels with forskolin led to activation of pCreb and Creb targets (FIG. 3B). In contrast, inhibition of Pka by H89 had the opposite effect, as it did in the long-term hair cycle experiments. The stimulation of canonical cAMP signaling occurred with a negligible effect on HFSC markers, but did coincide with an upregulation of PCNA (proliferation marker) in many parts of the epidermis (FIG. 3B) and HFSC bulge area. In addition, acute treatment with #44, cAMP, and forskolin also induced levels of transcript for canonical Creb targets as shown by cFos staining.

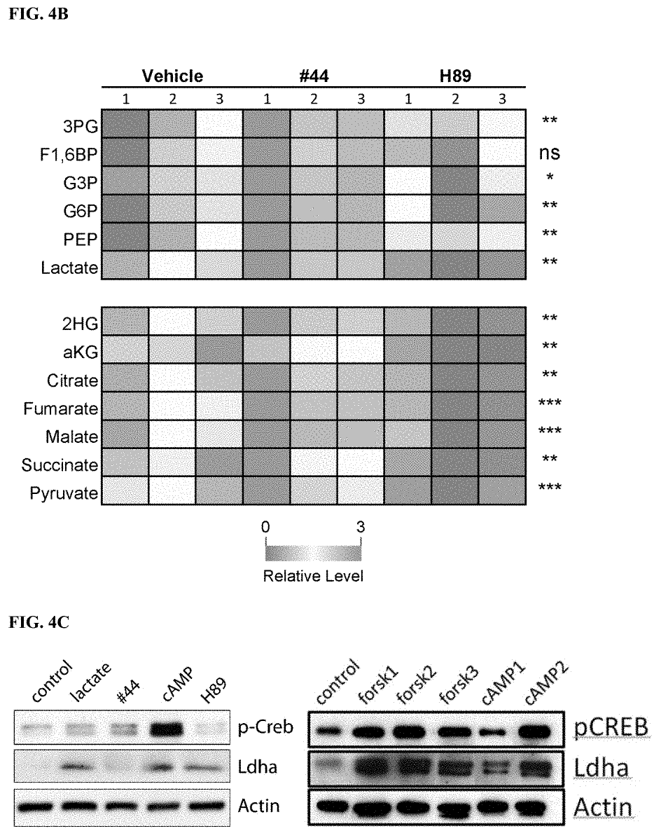

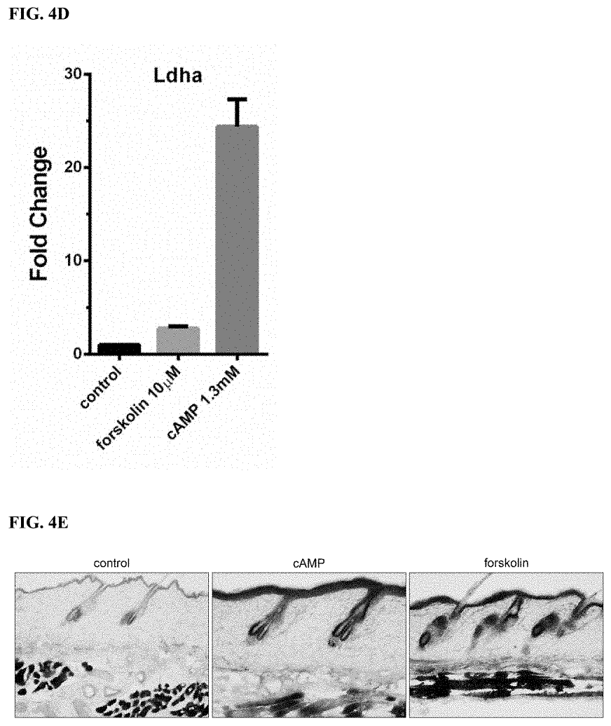

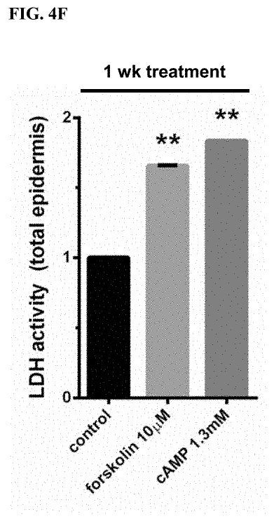

It was hypothesized that Creb could be driving proliferation due to an effect on cell metabolism as described in other systems in previous studies. In addition, it was shown that increasing glycolytic metabolism in HFSCs can promote their activation and accelerate the hair cycle. Applying several methods to measure the activity of a key metabolic enzyme, Lactate Dehydrogenase, we explored the possibility that Creb activation leads to increased transcription and translation of Ldh enzyme. We first used an activity assay for Ldh on cells isolated from epidermis treated with Pde inhibitors or cAMP. This assay showed that Pde inhibition has a robust effect on Ldh activity in treated skin, while Pka inhibition did not (FIG. 4A). This effect is also conserved regardless of sex (FIG. 4A). To more directly test the effect of elevated cAMP levels on metabolism, isolated treated epidermis samples were used for metabolomics analysis. This analysis demonstrated that most glycolytic metabolites were induced by Pde inhibition, as would be expected if Creb is able to stimulate the production of glycolytic enzymes (FIG. 4B), thereby increasing glycolysis in HFSCs, which in turn primed the HFSCs' activation. Western blotting for Ldha protein also showed an increase in protein production in response to Pde inhibition, cAMP treatment, or forskolin treatment (FIG. 4C). Furthermore, RT-PCR for Ldha transcript also showed an induction in epidermis isolated from skin treated with forskolin or cAMP (FIG. 4D). An LDH assay—used to assess lactate dehydrogenase activity in situ or via plate reader subjected to 475 nm absorbance measurements—showed acute stimulation of Creb-dependent signaling stimulated glycolytic activity, including Ldha activity, in HFSCs and in the IFE (FIG. 4E, FIG. 4F). Together, these data are consistent with the notion cAMP/Creb signaling can induce HFSC activation and the hair cycle through an upregulation of Ldh activity.

In one aspect, the present disclosure relates to a pharmaceutical composition, such as a composition suitable for topical administration to the skin, comprising:

a compound of formula I or a pharmaceutically acceptable salt thereof:

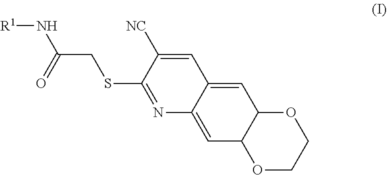

wherein R¹ is substituted or unsubstituted phenyl;
and a pharmaceutically acceptable excipient.

R¹ may be phenyl that is substituted or unsubstituted. In some embodiments, R¹ is substituted with 1, 2, 3, or 4 substituents. In some embodiments, the substituent is lower alkoxy. In some preferred embodiments, the alkoxy is methoxy or ethoxy. In some embodiments, the compound is 44, 61, or 62, as depicted in Table 1.

TABLE 1
Exemplary Compounds of the Present Invention

| | |
|---|---|
| 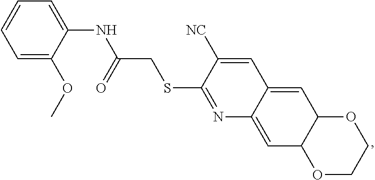 [SMILES: COc1ccccc1NC(=O)CSc1nc2c(cc1C#N)=CC1OCCOC1C=2] | 44 |
| 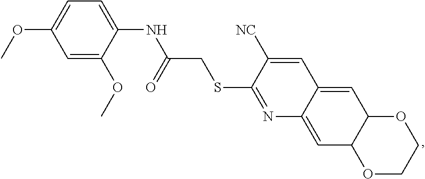 [SMILES: COc1ccc(NC(=O)CSc2nc3c(cc2C#N)=CC2OCCOC2C=3)c(OC)c1] | 61 |
| 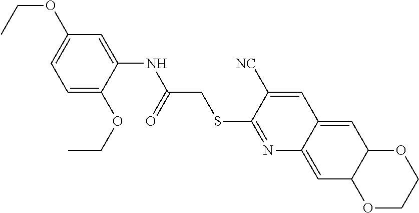 [SMILES: CCOc1ccc(OCC)c(NC(=O)CSc2nc3c(cc2C#N)=CC2OCCOC2C=3)c1] | 62 |

In another aspect, the present disclosure provides methods of inhibiting a phosphodiesterase inhibitor enzyme in a subject, comprising administering a compound of formula I or a pharmaceutically acceptable salt thereof:

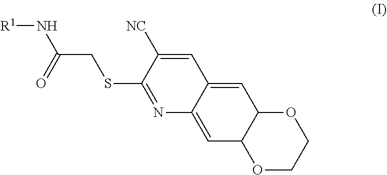

wherein R¹ is substituted or unsubstituted phenyl.

In yet another aspect, the present disclosure provides methods of promoting hair growth or treating a condition or disorder affecting hair growth in a subject, comprising administering (e.g., topically) a phosphodiesterase inhibitor to the subject. In certain embodiments, administering the phosphodiesterase inhibitor enzyme activates cAMP response element-binding protein (CREB or CREB-TF). In some embodiments, the phosphodiesterase inhibitor is a compound of formula I or a pharmaceutically acceptable salt thereof:

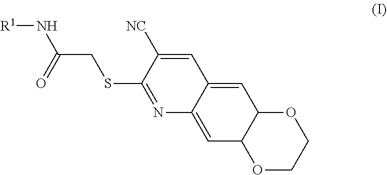

wherein R¹ is substituted or unsubstituted phenyl.

R¹ may be phenyl that is substituted or unsubstituted. In some embodiments, R¹ is substituted with 1, 2, 3, or 4 substituents. In some embodiments, the substituent is lower alkoxy. In some preferred embodiments, the alkoxy is methoxy or ethoxy. In some embodiments, the compound is 44, 61, or 62, as depicted in Table 1.

In some embodiments, the method is a method of promoting hair growth. In other embodiments, the method is a method for treating a condition or disorder affecting hair growth in a subject.

The condition or disorder affecting hair growth in the subject may be baldness or alopecia or a condition related thereto. For example, the condition or disorder may be androgenic alopecia, alopecia areata, alopecia universalis, alopecia totalis, ophiasis, traction alopecia, chignon alopecia, hypotricosis, telogen effluvium, lichen planopilaris, trichorrhexis nodosa, or folliculitis.

In certain embodiments, the compound is administered to a subject in need thereof. In certain embodiments, the compound is administered to a human subject.

In some embodiments, the phosphodiesterase enzyme is in the skin the subject. In some embodiments, the phosphodiesterase enzyme is in the dermis of the subject. In some preferred embodiments, the phosphodiesterase enzyme is in a hair follicle of the subject.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, l-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, l-pyroglutamic acid, salicylic acid, sebacic acid, stearic acid, succinic acid, sulfuric acid, l-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as alopecia or hair loss. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

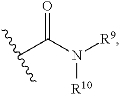

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

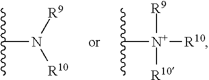

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

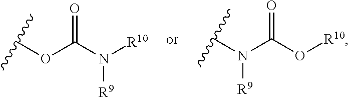

wherein R⁹ and R¹⁰ independently represent hydrogen or a hydrocarbyl group.

The term “carbocyclylalkyl”, as used herein, refers to an alkyl group substituted with a carbocycle group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H. The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═0 substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

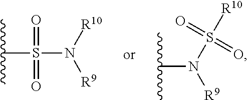

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

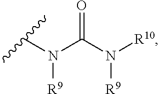

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Example 1: Defining the Role of cAMP/Creb Signaling in HFSC Activation

### Animals

Wildtype male and female 49-day old C57BL/6 mice were obtained from Jackson Laboratories for all topical experiments. All animals were maintained in UCLA's Division of Laboratory Medicine (DLAM)-approved pathogen-free barrier facilities and procedures were performed using protocols that adhere to the standards of the UCLA Animal Research Committee (ARC), Office of Animal Research Oversight (OARO), and the National Institutes of Health (NIH).

### Topical Inhibitor Treatments

Soluble cAMP and forskolin were purchased from a commercial vendor (Sigma B5386, Sigma F3917 respectively) along with PKA inhibitor H89 (Sigma 371963-M EMD Millipore). All drugs were suspended in DMSO and aliquoted at approximately 5 times IC50 or EC50 for the purposes of penetrating the epidermal barrier upon in vivo application. Aliquots were then mixed with the appropriate volume of Premium Lecithin Organogel (PLO) Base (Transderma Pharmaceuticals Inc.) and topically applied onto the shaved dorsal skin of 49 day old telogen-stage mice. Three experiment timepoints were used: acute treatment (two doses across 48 hours), 1 week treatment (three doses across five days) or long-term studies (one dose three times a week for approximately 1.5 weeks). Full-thickness dorsal skin was collected for histological analysis, cryosectioning, RNA isolation, and/or for epidermal stem cell isolation according to well-established FACS protocol.

### Hair Cycle Analysis

Hair cycles were assessed macroscopically by observational and photographic documentation. A scale was established to grade hair cycle stage: 1) telogen (pink, white skin when dorsally shaved); 2) pigmentation (broad blue/grey pigment spots/patches on shaved dorsal area); 3) hair growth (dark pigmentation coupled with small patches of fur); 4) anagen (dark, full patches of fur within dorsal area) in relation to number of doses and elapsed time of treatment. H&E images of all treatment conditions were imaged at 20× magnification to assess morphological changes in control and treated skin. Hair cycle stages were also evaluated by follicular morphology into respective telogen, telogen-to-anagen transition, or anagen categories.

### Histology and Immunohistochemistry

A set of full-thickness skin samples were obtained from post-mortem tissue harvesting, fixed overnight in 4% paraformaldehyde, and then dehydrated for paraffin embedding and slide generation; hematoxylin and eosin staining according to standard protocol. For immunohistochemistry, formalin-fixed, paraffin-embedded tissue slides were cleared and rehydrated through a series of ethanol washes. Antigen retrieval (20 min in pressure cooker, microwaved at 100 power) was performed with 10 mM citrate. Slides were incubated in hydrogen peroxide (30 min at 4 C) and then blocked with 10% goat serum/0.1% tween for 1 hour at room temperature. Primary antibodies were added and incubated overnight. The following antibodies were used—rabbit pCreb (Cell Signaling 9198, 1:800); rabbit Sox9 (Abcam 185966, 1:800); rabbit Ki67 sp6 (Abcam 16667, 1:50); rabbit PCNA (Cell Signaling 13110, 1:700); rabbit cFos (Abcam 7963, 1:100). Sections were washed the following day with 0.1% PBST and incubated with rabbit secondary HRP-labeled polymer (Dako) for 1 hour at room temperature, and then quickly washed with 0.1% PBST and PBS. AEC chromogen (Vector) was used for the colometric development reaction. Slides were then briefly counterstained with hemotoxylin, mounted with Faramount Aqueous Mounting Media (Dako) and sealed for subsequent visualization by light microscopy.

### Microscopy

Bright-field immunohistochemistry and hematoxylin/eosin-stained images were captured using an Olympus BX51 light microscope at 20× magnification or 40× using oil immersion.

### Ldh Plate Reader Assay

For the in situ assay, fresh tissue section slides of vehicle, or vehicle+the indicated treatment were briefly fixed in formalin and washed twice with PBS. For the plate reader assay, protein lysate was obtained from cell pellets and resuspended in RIPA buffer with HALT protease and phosphatase inhibitors (Thermo Scientific). Staining solutions for lactate dehydrogenase (Ldh) were prepared with Tris buffer pH 7.4, NBT (for in situ) or XTT formazan (for plate reader), NAD, PMS, substrate (lactate), and reagent-grade water and held at 37° C. until use. For in situ, solution was added directly onto tissue samples and incubated at 37° C. until appropriate saturation of Ldh enzyme signal was observed. Slides were then briefly washed in MilliQ water and counterstained with Brazilliant! nuclear stain (Anatech) for 3 minutes. Afterwards, slides were washed twice with PBS, mounted with glycerol, and sealed. For plate reader, solution was added directly onto lysate samples prepared in triplicate across a 96-well plate. A microplate reader held at 37° C. measured 457 nm absorbances every 3 minutes across a 3 minute period to assess enzyme kinetics, using a Synergy-MX plate reader (Biotek Instruments). Ldh activity was determined by calculating the change in slope of absorbance levels at all timepoints for each respective treatment.

### Metabolomics

The experiments were performed as described in (Miranda et al., 2017). Briefly, cells were washed with cold 150 mM ammonium acetate (pH 7.3) and a two-phase extraction was performed: addition of 400 uL cold 100% MeOH, 400 uL cold sterile H20, 10 nmol D/L-norvaline (internal standard), 400 uL of cold chloroform. After mixing and pelleting centrifugation at 4 C, the aqueous layer of the supernatant was moved to glass vials, desiccated under vacuum, and re-suspended in 70% acetonitrile. 5 ul of sample were injected onto a Luna NH2 (150 mm×2 mm, Phenomenex) column. Samples were analyzed by an UltiMate 3000RSLC (Thermo Scientific) coupled to a Q Exactive mass spectrometer (Thermo Scientific). The Q Exactive ran with polarity switching (+3.50 kV/−3.50 kV) in full scan mode with an m/z range of 65-975. Separation was performed using A) 5 mM NH4AcO (pH 9.9) and B) ACN. The gradient ran from 15% to 90% over 18 min, followed by an isocratic step for 9 min and reversal to the initial 15% A) for 7 min. Metabolites were quantified with TraceFinder 3.3 using accurate mass measurements (≤3 ppm) and retention times.

### RNA Extraction and RT-PCR

Total RNA was first extracted using a two-phase chloroform approach and further purified using the Zymo Direct-zol RNA MiniPrep Kit (Catalog No R2070). RNA was measured using a NanoDrop One Microvolume UV-Vis Spectrophotometer. The SuperScript III First-Strand Synthesis SuperMix (Invitrogen) was then used to generate cDNA for RT-PCR experiments subsequently performed on the Roche Lightcycler 480 II System.

### Western Blot

Protein lysate was obtained from whole epidermis cell pellets and resuspended in RIPA buffer with HALT protease and phosphatase inhibitors (Thermo Scientific). Protein concentration was quantified using the Pierce BCA protein assay. (Thermo Scientific). Prior to gel loading, each sample was mixed with Laemmli Buffer containing 5% BME (Bio-Rad) and boiled at 95° C. for 7 minutes. Approximately 15 ug of lysate was loaded into each well of a 4-12% NuPage Bis-Tris gel (Invitrogen) using 1× NuPage MOPS Running Buffer (Life Technologies). Gel was run at 80V for 1 hour to evenly stack, then 120V for 90 minutes until lanes reached the gel base. Gels were then transferred onto a nitrocellulose membrane using 1× NuPage Transfer Buffer (Life Technologies) and run at 370 A for 90 minutes on ice at room temperature. Membranes were blocked with 5% BSA/TBS+0.1% tween (TBST) and incubated overnight with primary antibody diluted in 5% BSA/TBST/milk. The following day, membranes were washed twice with TBST and then incubated with secondary HRP (Invitrogen) for 1 hour at room temperature. Membranes were washed once more, then visualized using SuperSignal Pico ECL Substrate (Thermo Sci) and developed onto film (Genessee). For re-use and re-probing of membranes, blots were washed and stripped at 37° C. for 15 minutes prior to re-blocking and overnight primary antibody incubation.

### Statistical Analysis

Data were analyzed and error bars represent standard error of the mean. An unpaired, two-tailed student's t-test determined significance, with values P<0.05 considered statistically significant, denoted by asterisks (*P<0.05; **P<0.01).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A pharmaceutical composition, such as a composition suitable for topical administration to the skin, comprising:
a compound of formula I or a pharmaceutically acceptable salt thereof: 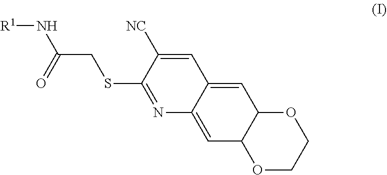
wherein R¹ is substituted or unsubstituted phenyl;
and a pharmaceutically acceptable excipient.

2. The pharmaceutical composition of claim 1, wherein R¹ is phenyl substituted with 1, 2, 3, or 4 lower alkoxy groups, such as methoxy or ethoxy.

3. The pharmaceutical composition of claim 1, wherein the compound is selected from the following compounds: 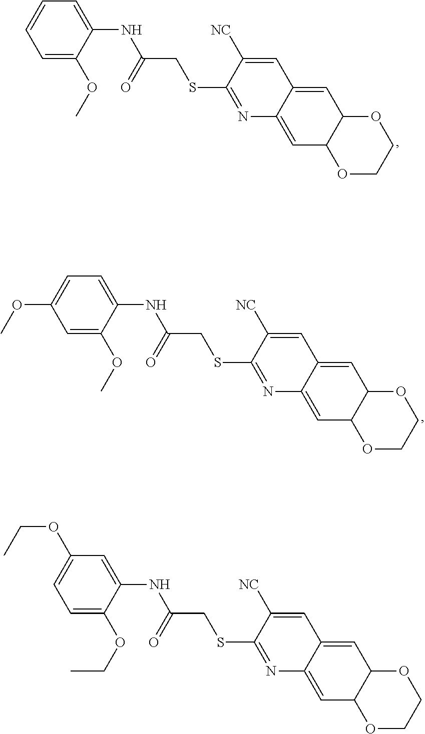 [SMILES: COc1ccccc1NC(=O)CSc1nc2c(cc1C#N)=CC1OCCOC1C=2 ; COc1ccc(NC(=O)CSc2nc3c(cc2C#N)=CC2OCCOC2C=3)c(OC)c1 ; CCOc1ccc(OCC)c(NC(=O)CSc2nc3c(cc2C#N)=CC2OCCOC2C=3)c1]
or a pharmaceutically acceptable salt thereof.

4. A method of inhibiting a phosphodiesterase enzyme in a subject, comprising administering a compound of formula I or a pharmaceutically acceptable salt thereof: 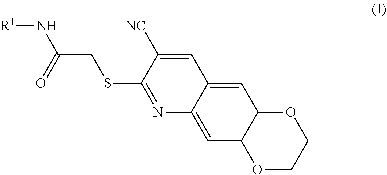
wherein R¹ is substituted or unsubstituted phenyl.

5. The method of claim 4, wherein R¹ is phenyl substituted with 1, 2, 3, or 4 lower alkoxy groups, such as methoxy or ethoxy.

6. The method of claim 5, wherein the compound is selected from the following compounds: 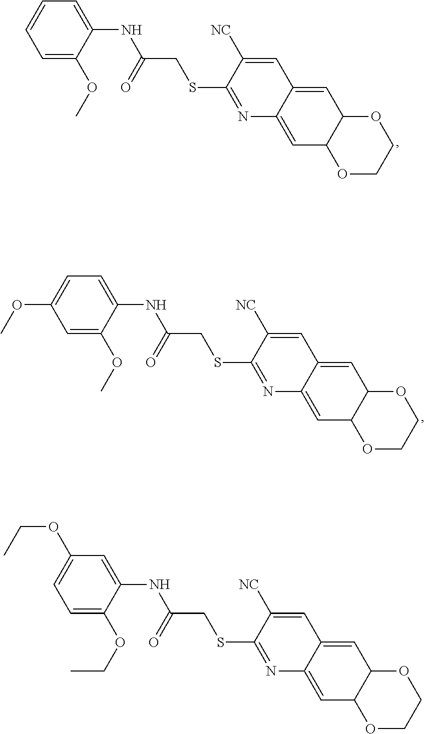 [SMILES: COc1ccccc1NC(=O)CSc1nc2c(cc1C#N)=CC1OCCOC1C=2 ; COc1ccc(NC(=O)CSc2nc3c(cc2C#N)=CC2OCCOC2C=3)c(OC)c1 ; CCOc1ccc(OCC)c(NC(=O)CSc2nc3c(cc2C#N)=CC2OCCOC2C=3)c1]
or a pharmaceutically acceptable salt thereof.

7. A method of promoting hair growth or treating a condition or disorder affecting hair growth in a subject, comprising administering a phosphodiesterase inhibitor to the subject.

8. The method of claim 7, wherein the phosphodiesterase enzyme is in a hair follicle of the subject.

9. The method of claim 7, wherein administering the phosphodiesterase inhibitor activates cAMP response element-binding protein.

10. The method of claim 7, wherein the phosphodiesterase enzyme inhibitor is a compound of formula I or a pharmaceutically acceptable salt thereof: 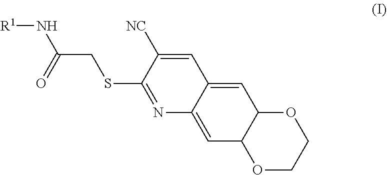
wherein R¹ is substituted or unsubstituted phenyl.

11. The method of claim 10, wherein R¹ is phenyl substituted with 1, 2, 3, or 4 lower alkoxy groups, such as methoxy or ethoxy.

12. The method of claim 10, wherein the compound is selected from the following compounds: 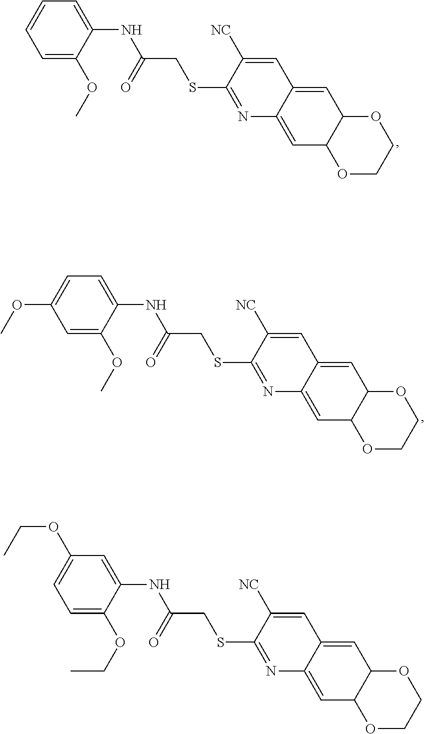 [SMILES: COc1ccccc1NC(=O)CSc1nc2c(cc1C#N)=CC1OCCOC1C=2 ; COc1ccc(NC(=O)CSc2nc3c(cc2C#N)=CC2OCCOC2C=3)c(OC)c1 ; CCOc1ccc(OCC)c(NC(=O)CSc2nc3c(cc2C#N)=CC2OCCOC2C=3)c1]
or a pharmaceutically acceptable salt thereof.

13. The method of claim 7, wherein the hair growth disorder is baldness or alopecia.

14. The method of claim 7, wherein the hair growth disorder is androgenic alopecia, alopecia areata, alopecia universalis, alopecia totalis, ophiasis, traction alopecia, chignon alopecia, hypotricosis, telogen effluvium, lichen planopilaris, trichorrhexis nodosa, or folliculitis.

15. The method of claim 7, wherein the phosphodiesterase inhibitor is administered topically.
